(19) United States
(12) Patent Application Publication
(10) Pub. No.: US 2024/0352432 A1
(43) Pub. Date: Oct. 24, 2024

(54) RAPID EXPRESSION AND PURIFICATION OF THERMOSTABLE PROTEINS INCLUDING TAQ POLYMERASE

(71) Applicant: Duke University, Durham, NC (US)

(72) Inventors: Michael Lynch, Durham, NC (US); Romel Menacho Melgar, Durham, NC (US); Tian Yang, Durham, NC (US)

(21) Appl. No.: 18/682,651

(22) PCT Filed: Aug. 11, 2022
(86) PCT No.: PCT/US2022/074823

§ 371 Date: Feb. 9, 2024

Related U.S. Application Data
(60) Provisional application No. 63/231,756, filed on Aug. 11, 2021.

(51) Int. Cl. C12N 9/12; C12N 1/06; C12N 9/22; C12N 9/36

(52) U.S. Cl. CPC C12N 9/1252; C12N 1/06; C12N 9/22; C12N 9/2462; C12Y 207/07007; C12Y 302/01017

(57) ABSTRACT

Genetically modified microorganisms enabling the simple, rapid production and purification of thermostable polymerases are described. Methods of rapid purification of a thermostable polymerase are described including the steps of autoinducible expression achieving high titers of soluble protein; autolysis of cells post harvest, autohydrolysis of host nucleotides through expression of a thermolabile endonuclease; and heat denaturation and precipitation of contaminating proteins. Polymerases are obtained with >95% purity, and are readily usable in standard PCR.

### CROSS-REFERENCE

This application is a National Stage Entry of PCT/US2022/74823, Aug. 11, 2022 which claims the benefit of U.S. Provisional Application No. 63/231,756, filed Aug. 11, 2021, which application is incorporated herein by reference in its entirety.

### FEDERALLY SPONSORED RESEARCH OR DEVELOPMENT

This invention was made with government support under DOE #EE7563; NCBC #2018BIG6503; and NIH #R61AI140485-01. The government has certain rights in the invention.

### FIELD OF THE INVENTION

This invention relates methods and compositions that provide an easy, readily accessible process for the routine production of high quality thermostable polymerases.

### BACKGROUND OF THE INVENTION

Research enzymes are required for numerous methodologies ranging from routine cloning, to protein purification and even in preparing samples for next generation sequencing. The costs of these reagents make up a significant portion of the costs of executing many experimental plans. DNA modifying enzymes, including thermostable enzymes, are ubiquitous reagents in molecular biology. Producing purified enzymes for molecular biology applications often requires the addition of purified nucleases and chromatographic separations to remove contaminating proteins as well as nucleic acids, which can affect use in downstream applications. The purification steps required to make “homemade” enzymes of necessary quality, have historically been a barrier. Even Taq polymerase, perhaps one of the most ubiquitous reagents can carry a hefty price tag. Although numerous methods have been reported for the expression and purification of these enzymes, they are not practical for many labs, requiring time and often specialized equipment such as a chromatography system.

### SUMMARY OF THE INVENTION

We describe herein a low-cost purification method for TAQ polymerase and Tth ligase representing a minimal set enabling both DNA amplification as well as DNA assembly. The method relies on 1) autoinducible expression achieving high protein titers, 2) heat induced autolysis and auto DNA/RNA hydrolysis via lysozyme and a mutant benzonase™, and 3) heat denaturation under reducing conditions to precipitate contaminating proteins including the mutant benzonase™. Enzymes are obtained at high purities and are readily usable in standard reactions. The method takes 40 minutes of hands-on time, does not require special equipment, expensive reagents, or affinity purification. This methodology is readily extensible to numerous additional proteins.

Thermostable proteins such as industrial enzymes or polymerases including Taq are ubiquitous reagents in virtually every lab performing molecular biology. Producing your own purified thermostable polymerases often requires chromatographic purification to remove not only unwanted proteins but importantly contaminating DNA which can affect downstream use, such as in PCR. Described herein is an easy, readily accessible method for the routine production of high quality thermostable polymerases as well as providing the microorganisms usable in the method.

Other methods, features and/or advantages is, or will become, apparent upon examination of the following Figures and detailed description. It is intended that all such additional methods, features, and advantages be included within this description and be protected by the accompanying claims.

### BRIEF DESCRIPTION OF THE DRAWINGS

The novel features of the invention are set forth with particularity in the claims. A better understanding of the features and advantages of the present invention will be obtained by reference to the following detailed description that sets forth illustrative embodiments, in which the principles of the invention are used, and the accompanying drawings of which:

FIG. 1 depicts a schematic of a precipitation based method for purification of a thermostable protein.

FIG. 2 depicts gel electrophoresis demonstrating purification of a TAQ polymerase according to the method of FIG. 1 and Table 1.

FIG. 3A-B: 3A) Taq expression and initial purification using E. coli strain DLF_R004 (lane 1, ladder; lane 2, empty vector control; lane 3, Taq expression; lane 4, Heat extract after 20 minutes at 70° C.). 3B) PCR product degradation using initial purified Taq preparations in comparison to commercial preparations. A PCR product made using commercial Taq (i) and initial Taq purifications (ii, from panel a, lane 4), immediately after PCR and after overnight incubation at 4° C.

FIG. 4 depicts nuclease activity (micro units) in lysates prepared from either strain DLF_R004 or DLF_R005 including: untreated lysates, lysates after 20 minutes at 60° C., and heat treated lysates in the presence of 0.5 M beta-mercaptoethanol (BME).

FIG. 5A-B depicts 5A) Optimization of nuclease removal via Design of Experiments. Nuclease activity measured after heat precipitation using 38 different precipitation conditions, each comprising different ‘Levels’ of beta-mercaptoethanol (0 to 1 M), temperature (60 to 80° C.), time (5 to 60 minutes), pH (6.0 to 9.0) and ammonium sulfate (0 to 1 M). The upper panel shows nuclease activity ranked from lowest to highest activity. The lower panel shows the precipitation condition for each result. 5B) Taq Purification using an optimal precipitation condition. Protein ladder (lane 1), Lysate of DLF_R005 expressing Taq (lane 2), Taq after optimal precipitation condition (lane 3, 80° C. for 60 minutes at pH 9.0 and 0.5 M beta-mercaptoethanol).

FIG. 6A-B depicts (6A) Protein release after thermolysis at different temperatures with and without Triton X-100. (6B) DNA removal after thermolysis and incubating at 37° C. (c) Contaminating DNA in Instant TAQ preparations made with freeze-thaw autolysis (lane 2) and thermolysis (lane 3) showing no contaminating DNA for thermolysis

FIG. 7A-C depicts PCR validation of commercial TAQ polymerase compared to Instant TAQ. (7A) Product specificity of commercial TAQ (dotted line) and instant TAQ (solid line) as shown by a qPCR melting curve. (7B) Agarose gel showing a PCR product made with commercial TAQ (top) and Instant TAQ (bottom). (7C) Amount of contaminating chromosomal DNA per units of TAQ of commercial TAQ compared to Instant TAQ as measured by qPCR. Commercial TAQ was purchased from New England Biolabs.

FIG. 8 depicts the Modeled Impact of key precipitation variables on nuclease removal. DoE predicted residual nuclease activity as a function of ammonium sulfate and BME concentrations, pH, temperature and heating time. For all 9 plots, pH is varied across the x-axis from 6 to 9. For all plots within the same column, the temperature is the same and fixed at 60° C., 70° C. or 80° C. for the left, middle and right column, respectively. For the 3 plots in the top row, ammonium sulfate is varied across the y-axis from 0 to 1 M while BME concentration is fixed at 0.5 M and heating time is fixed at 60 minutes. For the 3 graphs in the middle row, BME concentration is varied across the y-axis from 0 to 1 M while ammonium sulfate concentration is fixed at 1 M and heating time is fixed at 60 minutes. For the 3 graphs in the bottom row, heating time is varied across the y-axis from 5 to 60 minutes while ammonium sulfate concentration is fixed at 1 M and BME concentration is fixed at 0.5 M.

FIG. 9 depicts an SDS-PAGE demonstrating ligase purification using precipitation conditions according to one aspect of the invention. Protein ladder (lane 1), Lysate of DLF_R005 with a control empty vector (lane 2) or expressing ligase (lane 3), and purified ligase (lane 4).

FIG. 10 depicts an SDS-PAGE showing Instant TAQ purification using different reducing agents at various conditions: Protein ladder (lane 1), lysate (lane 2), 70° C. for 20 min (lane 3), 0.5 M B-Me added (lanes 4-6) with no heat (lane 4), 60° C. for 20 min (lane 4), 80° C. for 20 min (lane 5), 1 M B-Me added with 60° C. heat for 20 min (lane 6), 1 M B-Me added with 80° C. heat for 20 min (lane 7), 2 M B-Me added with 60° C. heat for 20 min (lane 8), 2 M B-Me added with 80° C. heat for 20 min (lane 9), 0.5 M DTT added (lane 10), 0.05 M DTT added with 60° C. for 20 min (lane 11), 0.5 M DTT added with 60° C. for 20 min (lane 12), 0.5 M TCEP added (lane 13), 0.05 M TCEP added with 60° C. for 20 min (lane 14), 0.5 M TCEP added with 60° C. for 20 min (lane 15).

FIG. 11 depicts protein release after thermolysis using DLF_R004 compared to a control strain (DLF_R003).

### DETAILED DESCRIPTION OF THE INVENTION

We describe herein engineered strains of E. coli enabling the simple, rapid production and purification of thermostable polymerases such as Taq and Phusion™. The process relies on 1) autoinducible expression achieving high titers of soluble protein; 2) autolysis of cells post harvest and autohydrolysis of host nucleotides through expression of a thermolabile endonuclease; and 3) optimized heat denaturation and precipitation of contaminating proteins. Polymerases are obtained with >95% purity, and are readily usable in standard PCR.

### Definitions

As used in the specification and the claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

The term “heterologous” is intended to include the term “exogenous” as the latter term is generally used in the art. With reference to the host microorganism's genome prior to the introduction of a heterologous nucleic acid sequence, the nucleic acid sequence that codes for the enzyme is heterologous (whether or not the heterologous nucleic acid sequence is introduced into that genome). As used herein, chromosomal and native and endogenous refer to genetic material of the host microorganism.

Species and other phylogenic identifications are according to the classification known to a person skilled in the art of microbiology.

Enzymes are listed here within, with reference to a UniProt identification number, which would be well known to one skilled in the art. The UniProt database can be accessed at http://www.UniProt.org/. When the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s) ”, “g” means gram(s), “μg” or “ug” means microgram(s) and “ng” means nanogram(s), “PCR” means polymerase chain reaction, “kDa” means kilodaltons, “g” means the gravitation constant, “bp” means base pair(s), “kbp” means kilobase pair(s), “% w/v” means weight/volume percent, “% v/v” means volume/volume percent, “rpm” means revolutions per minute, “HPLC” means high performance liquid chromatography, and “GC” means gas chromatography.

The ability to genetically modify a host cell is essential for the production of any genetically modified (recombinant) microorganism. The mode of gene transfer technology may be by electroporation, conjugation, transduction, or natural transformation. A broad range of host conjugative plasmids and drug resistance markers are available. The cloning vectors are tailored to the host organisms based on the nature of antibiotic resistance markers that can function in that host. Also, as disclosed herein, a genetically modified (recombinant) microorganism may comprise modifications other than via plasmid introduction, including modifications to its genomic DNA.

More generally, nucleic acid constructs can be prepared comprising an isolated polynucleotide encoding a polypeptide having enzyme activity operably linked to one or more (several) control sequences that direct the expression of the coding sequence in a microorganism, such as E. coli, under conditions compatible with the control sequences. The isolated polynucleotide may be manipulated to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well established in the art.

The control sequence may be an appropriate promoter sequence, a nucleotide sequence that is recognized by a host cell for expression of a polynucleotide encoding a polypeptide of the present invention. The promoter sequence may contain transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell. The techniques for modifying and utilizing recombinant DNA promoter sequences are well established in the art.

For various embodiments of the invention the genetic manipulations may include a manipulation directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected culture conditions. Genetic manipulation of nucleic acid sequences may increase copy number and/or comprise use of mutants of an enzyme related to product production. Specific methodologies and approaches to achieve such genetic modification are well known to one skilled in the art.

The methods disclosed herein often refer to microorganism and cells. To be clear, methods described in this disclosure refer generally to isolation of a thermostable protein from a microorganism which is a cell.

### Overview of Invention Aspects

In one aspect, the invention may include a genetically modified microorganism for producing a thermostable protein comprising: inducible lysozyme, an endonuclease, or a combination thereof; and inducible expression of a heterologous thermostable protein. In this microorganism, upon induction of the microorganism, the endonuclease accumulates in the periplasm.

In another aspect, upon induction of the microorganism the lysozyme accumulates in the cytoplasm.

In another aspect, the inducible lysozyme of the genetically modified microorganism is lambda lysozyme.

In another aspect, the endonuclease of the genetically modified microorganism is thermolabile.

In another aspect, the temperature required for endonuclease denaturation is less than the temperature of the thermostable protein.

In another aspect, the endonuclease of the genetically modified microorganism is from Serratia marcescens or Shewanella frigidimarina.

In another aspect, the thermostable protein of the genetically modified microorganism is a polymerase, reverse transcriptase, binding protein, therapeutic protein, DNA binding protein, nuclease, proteins for human consumption, proteases, amylases, lipases, mannanases, phytase, kinase, lactase, xylanase, carbohydrase.

In another aspect, the thermostable protein of the genetically modified microorganism is Taq polymerase, Phusion polymerase, Streptavidin, cellulase, Cas9, Cpf1, insulin.

In another aspect, the genetically modified microorganism is an E. coli microorganism.

In another aspect, the invention includes a method with a first step of: providing a genetically modified microorganism characterized by inducible lysozyme, an endonuclease, or a combination thereof; and inducible expression of a heterologous thermostable protein. The method may also include the step of inducing the microorganism; lysing the cells using a mechanical, chemical, physical, enzymatic method, or a combination thereof; incubating the lysate in autohydrolysis buffer to allow degradation of DNA and RNA by the endonuclease; adding salt to the lysate; heating the lysate at a temperature below that of the denaturing temperature of the thermostable protein; precipitating and removing contaminating protein, thereby producing a purified thermostable protein.

In another aspect, the method includes lysis of cell by exposing them to at least 0.1% detergent to disrupt the cell membrane.

In another aspect, the method includes heating a lysate to a temperature below that of the denaturing temperature of the thermostable protein, but above that of the endonuclease.

In another aspect, the method includes regulation of pH of the lysate selectively precipitates contaminating proteins. In another aspect, the pH of the lysate is modified to selectively precipitate contaminating proteins wherein the endonuclease is one contaminating protein.

In another aspect, the method may include a step where the endonuclease is separated from the thermostable protein using size filtration.

In another aspect, the method may include a step of adding ammonium sulfate salt to the lysate.

In another aspect, the thermostable protein to be isolated is a ligase, or in some aspects, a Taq ligase or Tth ligase.

In another aspect, a heating step of the methods may include the addition of a reducing agent. In some aspects the reducing agent is 2-mercaptoethanol, dithiothreitol or tris(2-carboxyethyl)phosphine.

In yet another aspect, a method of lysing a genetically modified microorganism, comprising the following steps: a. providing a genetically modified microorganism comprising: inducible a lysozyme enzyme, an endonuclease enzyme, or a combination thereof; and b. placing the genetically modified microorganism in a medium and under conditions that induce the lysozyme enzyme, an endonuclease enzyme, or a combination thereof within microorganism; c. harvesting the microorganism from the medium; d. resuspending the microorganism in a lysis buffer with or without detergent to form a mixture; e. heating the resuspended microorganism and lysis buffer mixture above 45° C. for a minimum of about thirty minutes to form a heated microorganism and lysis buffer mixture. The method is characterized by heating the microorganism and lysis buffer mixture results in lysis of the microorganism and/or removal of DNA from the mixture.

In another aspect, step d of the method is further comprises freezing the microorganism and lysis buffer mixture.

In another aspect, the method may include a step f. incubating the heated microorganism and lysis buffer mixture additionally at 37° C. for a minimum of about sixty minutes.

In some aspects of the method, the endonuclease enzyme is selectively removed from the mixture.

### Disclosed Embodiments Are Non-Limiting

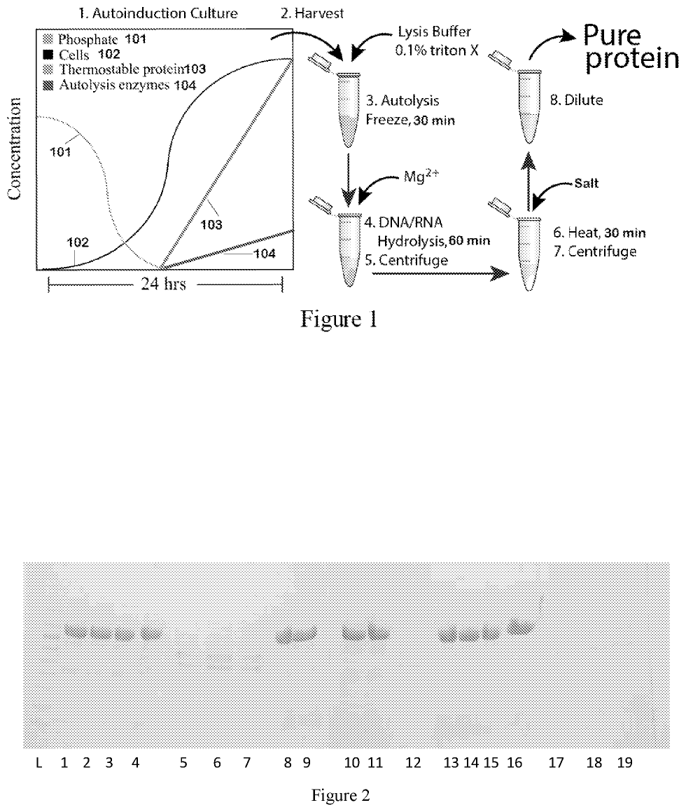

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping (such as metabolic pathway enzymes shown in a FIG. 1 unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. Moreover, when any range is described herein, unless clearly stated otherwise, that range includes all values therein and all sub-ranges therein.

Also, and more generally, in accordance with disclosures, discussions, examples and embodiments herein, there may be employed conventional molecular biology, cellular biology, microbiology, and recombinant DNA techniques within the skill of the art. Such techniques are explained fully in the literature. See, e.g., Sambrook and Russell, “Molecular Cloning: A Laboratory Manual,” Third Edition 2001 (volumes 1-3), Cold Spring Harbor Laboratory Press, Cold Spring Harbor, N.Y.; Animal Cell Culture, R. I. Freshney, ed., 1986. These published resources are incorporated by reference herein.

The following published resources are incorporated by reference herein for description useful in conjunction with the invention described herein, for example, methods of industrial bio-production of chemical product(s) from sugar sources, and also industrial systems that may be used to achieve such conversion (Biochemical Engineering Fundamentals, 2^{nd} Ed. J. E. Bailey and D. F. Ollis, McGraw Hill, New York, 1986, e.g. Chapter 9, pages 533-657 for biological reactor design; Unit Operations of Chemical Engineering, 5^{th} Ed., W. L. McCabe et al., McGraw Hill, New York 1993, e.g., for process and separation technologies analyses; Equilibrium Staged Separations, P. C. Wankat, Prentice Hall, Englewood Cliffs, NJ USA, 1988, e.g., for separation technologies teachings).

All publications, patents, and patent applications mentioned in this specification are entirely incorporated by reference herein.

### EXAMPLES

The examples herein provide some examples, not meant to be limiting. All reagents, unless otherwise indicated, are obtained commercially. Species and other phylogenic identifications are according to the classification known to a person skilled in the art of microbiology, molecular biology and biochemistry.

### Example 1: Precipitation Based Purification of a Thermostable Protein

Referring to FIG. 1, a schematic for preparation of a purified thermostable protein using a precipitation-based purification method is described. In 1, it can be seen that phosphate depletion induces expression of the target thermostable protein (eg TAQ polymerase) and autolysis enzymes (lysozyme and benzonase). 2. The cells are harvested and 3. lysed using 0.1 Triton X-100. 4. The lysate is incubated in autohydrolysis buffer for 60 min to degrade DNA and RNA and 5. Centrifuged to remove insoluble debris. 6. The lysate is heated and 7. contaminating proteins are removed by centrifugation to obtain 8. pure protein.

Table 1 provides specific experimental detail for the process outlined in FIG. 1. The results for the experimental outcome of these various experiments is found in the gel electrophoresis of FIG. 2 for purification of TAQ polymerase using a heat and precipitation step.

TABLE 1
Experimental Design

| Lane | Salt (M) | Temp (° C.) | Time (min) |
|---|---|---|---|
| 1 | 0 | 60 | 60 |
| 2 | 0 | 60 | 60 |
| 3 | 0 | 60 | 10 |
| 4 | 1 | 60 | 10 |
| 5 | 2 | 60 | 35 |
| 6 | 2 | 60 | 10 |
| 7 | 2 | 60 | 60 |
| 8 | 0 | 72.5 | 10 |
| 9 | 1 | 72.5 | 35 |
| 10 | 1 | 72.5 | 35 |
| 11 | 1 | 72.5 | 35 |
| 12 | 2 | 72.5 | 60 |
| 13 | 0 | 85 | 35 |
| 14 | 0 | 85 | 10 |
| 15 | 0 | 85 | 60 |
| 16 | 1 | 85 | 60 |
| 17 | 2 | 85 | 10 |
| 18 | 2 | 85 | 10 |
| 19 | 2 | 85 | 60 |

As a first step, 1) autoinduction of heterologous thermostable protein expression occurs upon phosphate depletion, together with 2) the induction of a lysozyme and/or a broad specificity endonuclease, which enable the autolysis and autohydrolysis of DNA/RNA and further the simultaneous heat denaturation and precipitation of contaminants enabling purification of thermostable proteins. When combined together as illustrated in FIG. 1, these approaches enable autoinducible protein expression, autolysis and autohydrolysis and rapid purification. As can be seen in FIG. 2, some preparations contained residual contaminating non-specific thermostable endonuclease (S. marcescens nucA, benzonase™) activity, which is not compatible with downstream PCR applications without further purification. The non-specific DNA/RNA endonuclease from S. marcescens, requires very harsh conditions for complete inactivation. Given these limitation, we demonstrate the utility of more thermolabile non-specific DNA/RNA endonuclease. A subsequent simple purification comprising heat denaturation followed by precipitation of contaminating proteins including the thermolabile endonuclease results in polymerases which are readily usable in standard PCRs.

### Example 2: Optimization the Process for Taq DNA Polymerase

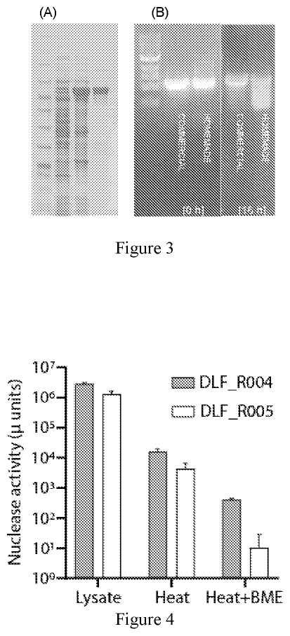

Firstly, to express Taq, we leveraged strains, expression constructs and protocols we have recently reported for the tightly controlled, low phosphate autoinduction of heterologous protein, as well as autolysis and autohydrolysis of DNA/RNA. Specifically, Taq was cloned into an expression construct driven by the robust, low phosphate inducible E. coli yibD gene promoter. This plasmid was transformed into strain DLF_R004 (Genotype: F-, λ-, Δ(araD-araB)567, lacZ4787(del)(::rrnB-3), rph-1, Δ(rhaD-rhaB)568, hsdR51, ΔackA-pta, ΔpoxB, ΔpflB, ΔldhA, ΔadhE, ΔiclR, ΔarcA, ΔompT::yibDp- [symbol] R-nucA-apmR), which has been engineered for the low phosphate induction of a periplasmic nuclease (S. marcescens nucA, benzonase™) and cytoplasmic Lambda phage lysozyme. After the disruption of the membrane in the presence of 0.1% triton-X, lysozyme degrades the peptidoglycan cell wall and the nuclease degrades residual cellular DNA and RNA. Cell growth and autoinduction of Taq expression is performed in autoinduction broth as described. Using this system, Taq was expressed to >35% of the total cellular protein approaching titers of >900 mg/L in shake flasks as seen in FIG. 3A.

With successful autolysis and auto DNA/RNA autohydrolysis, we next turned to the further purification of Taq. Initial attempts at Taq purification relied on simply heating lysates to 70° C. for 20 minutes followed by centrifugation to denature and precipitate contaminating proteins. These original preparations contained residual contaminating non-specific thermostable endonuclease (S. marcescens nucA) activity, leading to the degradation of DNA over time (FIB 3B).

### Example 3: Removal of Residual Benzonase

This contaminating activity of FIG. 3B is of course not compatible with downstream applications, including, in the case of Taq, routine PCR. As benzonase has crucial disulfide bonds required for correct folding and activity, we investigated the potential of adding reducing agents during the heat denaturation step to more completely inactivate and precipitate residual benzonase. The addition of 0.5M beta-mercaptoethanol during heat denaturation, greatly improved, but did not eliminate, contaminating endonuclease removal, as shown in FIG. 4. Based on this success, we constructed a new host strain, DLF_R005 (Genotype: F-, λ-, Δ(araD-araB)567, lacZ4787(del)(::rrnB-3), rph-1, Δ(rhaD-rhaB)568, hsdR51, ΔackA-pta, ΔpoxB, ΔpflB, ΔldhA, ΔadhE, ΔiclR, ΔarcA, ΔompT::yibDp- [symbol] R-nucA-apmR), identical to DLF_R004, wherein the wildtype dimeric benzonase was replaced with a monomeric variant. This variant is not only active as a monomer but has a lower melting temperature, making it a better candidate for thermal inactivation under reducing conditions. Importantly, strain DLF_R005 also enabled further reductions in nuclease activity (FIG. 4).

### Example 4: Design of Experiments (DoE) Methodology

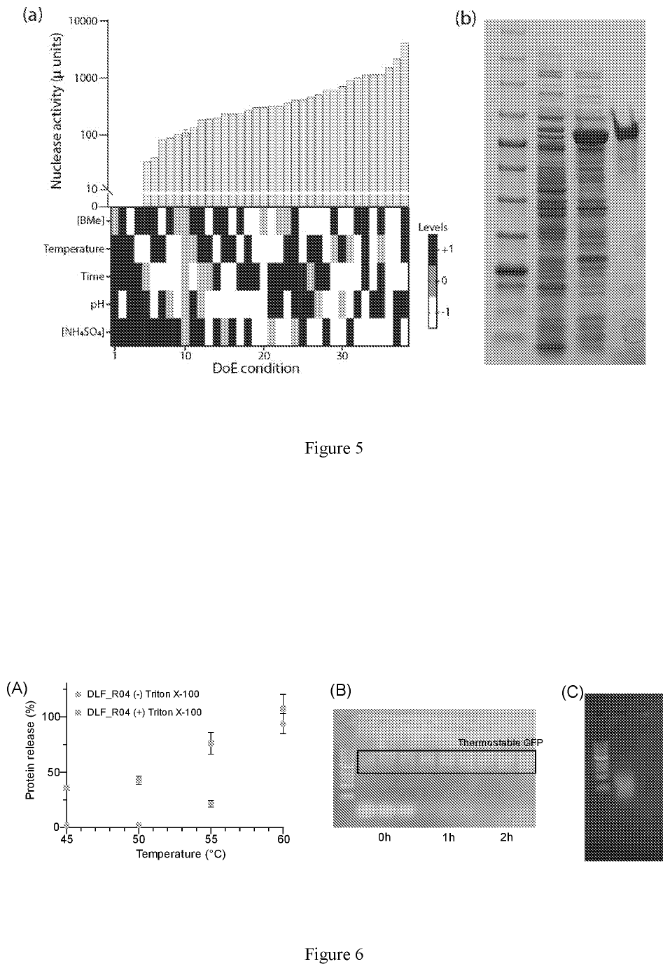

Building upon the success of the monomeric benzonase with reducing conditions, we then further optimized precipitation conditions using standard design of experiments (DoE) methodology as illustrated in FIG. 5. The temperature, salt concentration, pH and BME concentrations during the precipitation were varied and a model built to understand the relationship between these key variables and nuclease removal. As FIG. 5 demonstrates, a range of conditions lead to an optimal precipitation, with the monomeric benzonase being essentially completely removed.

### Example 5: Comparison of Instant Taq and Commercial Taq Preparations

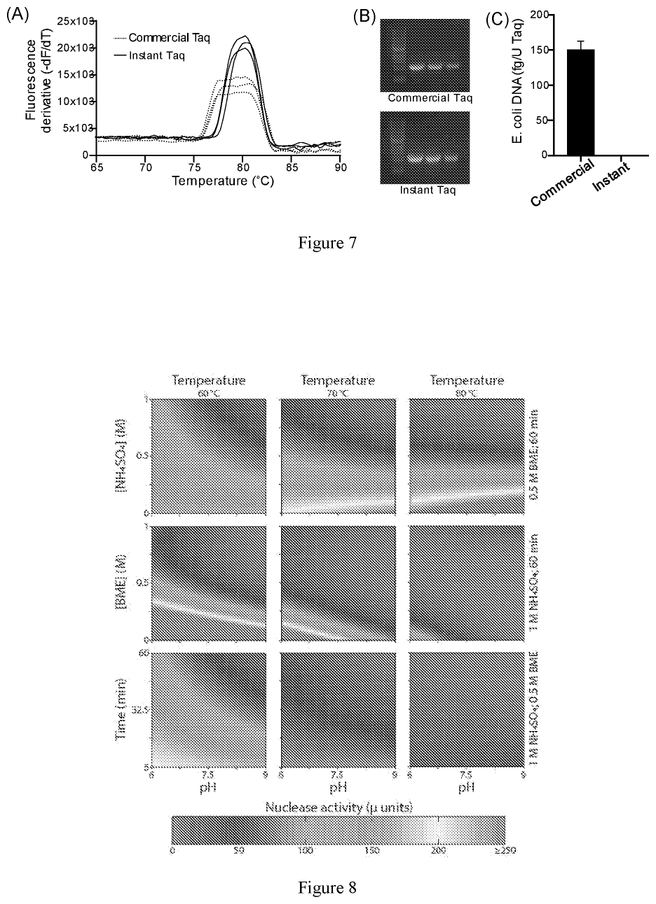

We proceeded to validate the performance of these ‘Instant’ Taq preparations in routine PCR compared to commercial Taq. While performing these tests, we noticed residual contaminating DNA in the Instant Taq preparations as illustrated in FIG. 6. We hypothesized this was due to the benzonase creating short oligonucleotides while degrading DNA that can then be extended by Taq polymerase after annealing to each other. We developed an alternative lysis protocol whereby we heat the cells at 60° C. for 1 hour after freezing to minimize the annealing of short oligonucleotides followed by incubation at 37° C. to remove the contaminating DNA. We further characterized this new lysis method, ‘thermolysis’ using DLF_R004 and found protein release did not require a freeze step. Optimal protein release using thermolysis, as shown in FIG. 6, occurred at 60° C. and did not need detergent in the lysis buffer. Contaminating DNA was not observed after 2 hours of incubation at 37° C. using agarose gel electrophoresis. With the success of eliminating contaminating DNA, we validated the activity of Instant TAQ and compared it to commercial TAQ. We found Instant TAQ performed equally or better than commercial TAQ, as shown in FIG. 7.

### Example 6: Applications to Additional Proteins

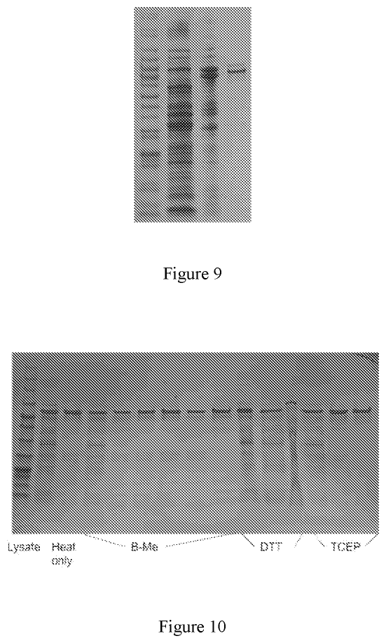

With the success of strain DLF_R005, enabling the production of Taq, we turned to assess the broader suitability of this approach for other proteins. Based on the DoE experiments, a model can built, predicting nuclease removal as a function of key precipitation variables. A graphical overview of this model is given in FIG. 8. Importantly, numerous combinations of temperature, pH, salt concentration and BME concentrations enable effective removal of nuclease activity. This allows for extensibility of this methodology to numerous proteins of interest, with varied solubility and isoelectric points and temperature stabilities (at least greater than 60 degrees Celsius). We sought to validate this approach for the “instant” purification of Tth Ligase. The same methodology was used to successfully express and purify Tth ligase as demonstrated in FIG. 9. Both major bands correspond to purified ligase (adenylated vs non-adenylated).

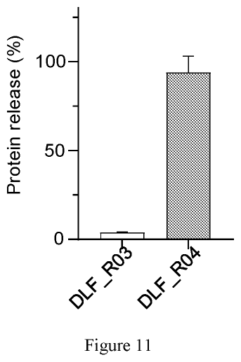

FIG. 10 demonstrates by SDS-PAGE instant TAQ purification with different reducing agents at various conditions. FIG. 11 demonstrates the effective protein release after thermolysis with the DLF_4 strain as compared to a control strain DLF_R003.

One skilled in the art will readily appreciate that the present disclosure is well adapted to carry out the objects and obtain the ends and advantages mentioned, as well as those inherent therein. The present disclosure described herein are presently representative of preferred aspects, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A genetically modified microorganism for producing a thermostable protein comprising:
an inducible lysozyme enzyme, an inducible endonuclease enzyme, or a combination thereof; and
inducible expression of a heterologous thermostable protein,
wherein, upon induction of the microorganism, the endonuclease accumulates in the periplasm.

2. The genetically modified microorganism of claim 1, wherein further upon induction the lysozyme accumulates in the cytoplasm.

3. The genetically modified microorganism of claim 1, wherein the lysozyme is lambda lysozyme and the endonuclease is thermolabile.

4. (canceled).

5. The genetically modified microorganism of claim 1, wherein the temperature of endonuclease denaturation is less than the temperature of the thermostable protein.

6. The genetically modified microorganism of claim 1, wherein the endonuclease is from Serratia marcescens or Shewanella frigidimarina.

7. The genetically modified microorganism of claim 1, wherein the thermostable protein is a polymerase, reverse transcriptase, binding protein, therapeutic protein, DNA binding protein, nuclease, proteins for human consumption, proteases, amylases, lipases, mannanases, phytase, kinase, lactase, xylanase, carbohydrase, Taq polymerase, Phusion polymerase, Streptavidin, cellulase, Cas9, Cpf1, or insulin.

8. (canceled).

9. The genetically modified microorganism of claim 1, wherein the microorganism is an E. coli microorganism.

10. A method of purifying a thermostable protein of claim 1, comprising the following steps:
providing a genetically modified microorganism comprising:
inducible lysozyme, an endonuclease, or a combination thereof; and
inducible expression of a heterologous thermostable protein;
inducing the microorganism;
lysing the cells using a mechanical, chemical, physical, enzymatic method, or a combination thereof;
incubating the lysate in autohydrolysis buffer to allow degradation of DNA and RNA by the endonuclease;
adding salt to the lysate;
heating the lysate at a temperature below that of the denaturing temperature of the thermostable protein;
precipiting and removing contaminating protein, thereby producing a purified thermostable protein.

11. The method of claim 10, wherein the cells are lysed by exposing them to at least 0.1% detergent to disrupt the cell membrane.

12. The method of claim 10, wherein the lysate is heated at a temperature below that of the denaturing temperature of the thermostable protein, but above that of the endonuclease.

13. The method of claim 10, wherein the pH of the lysate selectively precipitates contaminating proteins.

14. (canceled).

15. The method of claim 10, wherein the endonuclease is separated from the thermostable protein using size filtration.

16. The method of claim 10, wherein the salt added to the lysate is ammonium sulfate.

17. The method of claim 10, wherein the endonuclease enzyme is selectively removed from the lysate whereas the thermostable protein remains in solution and wherein the endonuclease enzyme is removed from the lysate by a combination of heat denaturation and precipitation.

18. (canceled).

19. The method of claim 10, wherein the thermostable protein is a Taq ligase.

20. (canceled)

21. The method of claim 10, wherein the heating step further comprises addition of a reducing agent during the heating and precipitation steps and the reducing agent is 2-mercaptoethanol, dithiothreitol or tris(2-carboxyethyl)phosphine.

22. (canceled).

23. A method of lysing a genetically modified microorganism, comprising the following steps:
a. providing a genetically modified microorganism comprising:
inducible a lysozyme enzyme, an endonuclease enzyme, or a combination thereof; and
b. placing the genetically modified microorganism in a medium and under conditions that induce the lysozyme enzyme, an endonuclease enzyme, or a combination thereof within microorganism;
c. harvesting the microorganism from the medium;
d. resuspending the microorganism in a lysis buffer with or without detergent to form a mixture;
e. heating the resuspended microorganism and lysis buffer mixture above 45° C. for a minimum of about thirty minutes to form a heated microorganism and lysis buffer mixture;
wherein heating the microorganism and lysis buffer mixture results in lysis of the microorganism and/or removal of DNA from the mixture.

24. The method of claim 23, wherein step d further comprises freezing the microorganism and lysis buffer mixture.

25. The method of claim 23, further comprising:
f. incubating the heated microorganism and lysis buffer mixture additionally at 37° C. for a minimum of about sixty minutes.

26. The method of claim 23, wherein the endonuclease enzyme is selectively removed from the mixture.
